(19) United States
(12) Patent Application Publication
(10) Pub. No.: US 2025/0364103 A1
(43) Pub. Date: Nov. 27, 2025

(54) STORAGE CABINET

(71) Applicant: Intrinsiq Specialty Solutions, Inc., Carrollton, TX (US)

(72) Inventors: Robert Neff, Villanova, PA (US); Jimmy Saunders, Dothan, AL (US); Raafat Ehmood, Yardley, PA (US); Joseph Braverman, Philadelphia, PA (US); Abigail Spraker, Morton, PA (US); Samuel Dorvil, Pembroke Pines, FL (US); Ethan White, Hummelstown, PA (US); Henry Steinberg, Mount Laurel, NJ (US); Anthony Mangine, Downingtown, PA (US); Nathan Godshall, Lafayette Hill, PA (US)

(21) Appl. No.: 19/004,628

(22) Filed: Dec. 30, 2024

Related U.S. Application Data
(60) Provisional application No. 63/651,802, filed on May 24, 2024.

(51) Int. Cl. G16H 20/13; A61J 7/00; G06Q 10/087

(52) U.S. Cl. CPC G16H 20/13; A61J 7/0076; G06Q 10/087

(57) ABSTRACT

A modular medication storage cabinet is provided having a plurality of storage units. Each storage unit includes an outer housing having a front opening and a front panel interconnected to the outer housing via a set of drawer sliders that enable the front panel to be moved in its entirety along a path of travel extending forward of the front opening from a closed position to an open position spaced forward of the front opening. A plurality of shelves is integrated on a rear side of the front panel at different heights such that, when the front panel is in the open position, the shelves are located exterior of the outer housing. Each of the shelves extend longitudinally relative to the path of travel of the front panel and define a plurality of compartments in which medicine or medical supplies are stored.

### CROSS REFERENCE TO RELATED APPLICATIONS

This application claims the benefit under 35 USC § 119 (e) of U.S. Provisional Patent Application No. 63/651,802 filed May 24, 2024.

### BACKGROUND

The present disclosure relates to storage cabinets for use with inventory management systems. As an example, the storage cabinets may be provided in healthcare environments, such as specialty physician practices. Alternatively, the storage cabinets may be used in other industries for other goods.

By way of example, medication inventory management systems are used in healthcare environments in order to administer the inventory and distribution of medication and other medical supplies to patients. Medications may be retrieved from a medication storage and/or dispensing cabinet or workstation, placed in patient specific containers, transported to the patient care areas, and administered as prescribed. The cabinets may serve as a workstation from which healthcare professionals can readily access medications or other medical supplies to be administered to the patients under their care.

A typical medication storage and dispensing workstation may comprise a cabinet having a plurality of storage locations, such as drawers, shelves, trays, cassettes, bins, or the like. The storage locations are stocked with individual medications and/or medication doses, or other medical supplies. The contents of the workstations are inventoried and the distribution of medications and medical supplies is carefully controlled.

Access to the medication storage and dispensing cabinet or workstation is typically limited and can be gained only by healthcare professionals with appropriate credentials. A computer-based user interface may enable access to the cabinet or workstation and may be used to record inventory and distribution of medications and/or medical supplies from the cabinet. For instance, after dispensing a medication, a healthcare worker may record a patient's usage into the inventory system and may return any leftover medications to the cabinet. The inventory management system may automatically update and record each transaction.

Such an automated system eliminates the time required to search for stored medication, as a correct drawer and/or compartment of a cabinet may automatically unlock and/or open. Such a system may also eliminate the need for human supervision of transactions, with inventory management system automatically recording each transaction and alerting when the cabinet requires refilling.

### SUMMARY

According to an embodiment, a modular storage cabinet is provided that includes a plurality of storage units. Each of the storage units includes an outer housing, defining a storage space therein and having a front opening, and a front panel interconnected to the outer housing via a set of drawer sliders that enable the front panel to be moved in its entirety along a path of travel extending forward of the front opening from a closed position in which the front panel closes the front opening to an open position spaced forward of the front opening. A plurality of shelves is integrated on a rear side of the front panel at different heights such that, when the front panel is in the closed position, the shelves are housed entirely within the outer housing and, when the front panel is in the open position, the shelves are located exterior of the outer housing. Each of the shelves extends longitudinally relative to the path of travel of the front panel and, when the front panel is in the open position, each of the plurality of shelves is open and accessible along both sides of the path of travel. A plurality of compartments is provided in each of the shelves in which medicine or medical supplies are stored.

According to another embodiment, a modular medication storage cabinet is provided for use with a computer controlled medication inventory management system for administering the inventory and distribution of medicines and medical supplies in a healthcare environment. The modular cabinet comprises a plurality of storage units including a pair of upper storage units and a pair of lower storage units. A controller unit is supported on the pair of lower storage units and directly beneath the pair of upper storage units. The controller unit has a height less than a height of the storage units and includes a forward extending open work surface. A battery unit is located behind the pair of lower storage units such that the lower units extend forward of the upper storage units and underneath the work surface. At least one of the panels includes a user interface of the inventory management system.

According to a table-top embodiment, a medication storage cabinet for use with a computer controlled medication inventory management system for administering the inventory and distribution of medicines and medical supplies in a healthcare environment is provided. The cabinet includes a plurality of storage units including a pair of upper storage units and a controller unit directly beneath the pair of upper storage units. The controller unit has a height less than a height of the storage units and includes a forward extending open work surface and at least one of a printer for printing labels or receipts, a scanner, or a limited access drawer for narcotics or high value medical products. At least one the front panels includes a user interface of the inventory management system.

### BRIEF DESCRIPTION OF THE DRAWINGS

The foregoing and other objects, features and advantages of the embodiments disclosed herein should become apparent from the following description when taken in conjunction with the accompanying drawings.

FIG. 1 is a perspective view of an embodiment of a cabinet according to an embodiment.

FIG. 2 is a perspective view of the cabinet of FIG. 1 including bins according to an embodiment.

FIG. 3 is a perspective view of the cabinet of FIG. 1 with one of the units of the cabinet in an open condition according to an embodiment.

FIG. 4 is a perspective view of compartments on a shelf of the cabinet of FIG. 3 according to an embodiment.

FIGS. 5A-5F are perspective views of different embodiments of beltway or controller units for the cabinet of FIG. 1.

FIG. 6 is an exploded perspective view of a modular cabinet assembly according to an embodiment.

FIG. 7 is a perspective view of a tabletop cabinet according to an embodiment.

FIG. 8A is a perspective view of a cabinet having bins and lights indicating drug location according to an embodiment.

FIG. 8B is a perspective view of a cabinet have a lockable bin in a locked condition, and FIG. 8C is a perspective view of the cabinet of FIG. 8A in which the lockable bin is an unlocked condition.

### DETAILED DESCRIPTION

For simplicity and illustrative purposes, the principles of the embodiments are described by referring mainly to examples thereof. In the following description, numerous specific details are set forth in order to provide a thorough understanding of the embodiments. It will be apparent however, to one of ordinary skill in the art, that the embodiments may be practiced without limitation to these specific details. In some instances, well known devices, apparatus, methods and structures have not been described in detail so as not to unnecessarily obscure the embodiments.

According to some embodiments disclosed herein, modular storage cabinets are provided so as to provide a local area to store multiple items required and/or used during daily tasks, such as performed within a healthcare environment. Such local areas may be defined within relatively small spaces. Accordingly, the cabinets according to some embodiments include an integrated workspace or work surface so as to allow items being removed or returned to the cabinet to be placed close to the cabinet and so as to prevent back-and-forth to a nearest available counter which may otherwise be required. Such an integrated workspace or work surface necessarily results in an increase in healthcare worker productivity and a reduction in foot traffic that would otherwise be required.

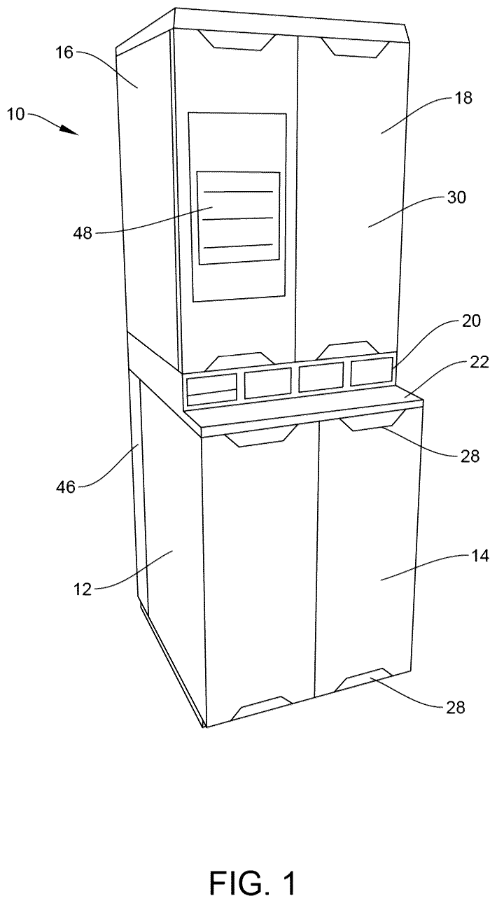

FIG. 1 provides an example of an embodiment of a medication storage cabinet 10 having an integrated workspace. The cabinet 10, in this embodiment includes four separate storage units, 12, 14, 16, and 18. Storage units, 12 and 14, are side-by-side lower units, and storage units, 16 and 18, are side-by-side upper units. A so-called beltway or controller unit 20 is located between the upper and lower units. The lower units, 12 and 14, extend further outward than the upper units, 16 and 18, and the beltway or controller unit 20 includes a front open workspace or work surface 22. Accordingly, regardless of which storage unit is opened, at least a part of the workspace or work surface 22 will be available to place items having just been removed or to be returned to the cabinet 10.

As shown in FIG. 1, the cabinet may include a computer-based user interface 48 that may provide part of an automated inventory management system. By way of example, a computer operating under the direction of a medication inventory management software application program may control the display of the user interface 28 and access to and operation of the cabinet 10. For example, the interface 28 may be used to permit access to a particular storage unit, enter patient information, record transactions, update inventory, or the like. Further, a back unit 46 of the cabinet 10 may provide a space for a battery pack, battery back-up modules, or other components such as computer hardware, or the like.

Access to each of the storage units may be limited to properly credentialed healthcare professionals by facility protocols. A healthcare professional may gain access to the medication storage units according to established protocols of the facility. Once access is obtained, the healthcare professional can retrieve medication(s) or other medical supplies from the storage units as they are needed or in order to fulfill the prescription requirements of individual patients which are under the care of the healthcare professional.

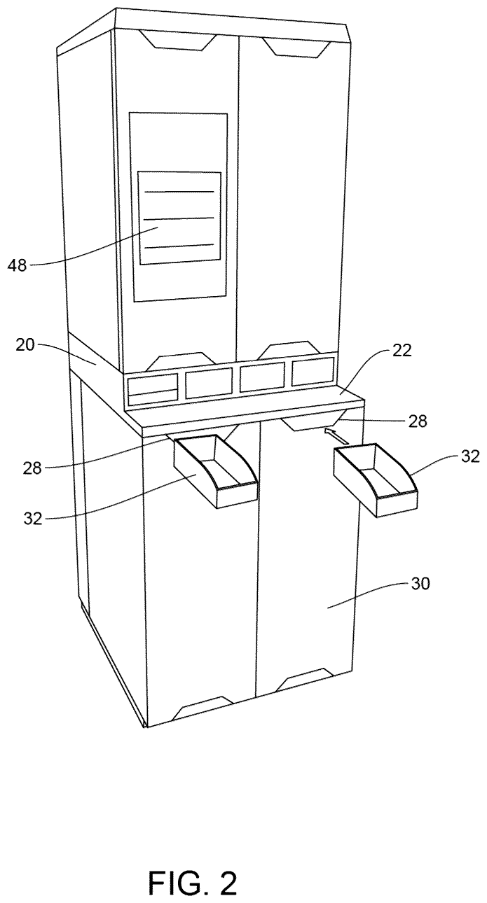

Each of units of the cabinet include a front panel 30. As best shown in FIG. 2, a cabinet may be provided with integrated bin hangers 28 built into top edges of the front panels 30 of at least the lower storage units. As an example, the hangers 28 may be in the form of trapezoidal cut-outs or recesses in the top edges of the front panels 30 which may also function as drawer handles. The hangers 28 provide the ability to hang bins 32 on the drawer handles. Thus, items dispensed for a patient may be placed in the bin 32, and then the bin may be used to carry the items to a pharmacy area or the like for mixing. Bins 32 may be the same or similar bins (i.e., size, shape, capacity, etc.) that may be located within the cabinet allowing for seamless exchanges from inside the cabinet to outside the cabinet and vice versa. As shown in FIG. 2, a healthcare worker has at least a portion of the workspace or work surface 22 as well as a bin 32 available for use adjacent to the cabinet.

Referring to the cabinet 10 shown in FIG. 1, the hangers 28 (or recessed edges) may be included at the top and bottom edges of every unit. This enables ease of manufacture since the same door can be installed on any unit in any orientation.

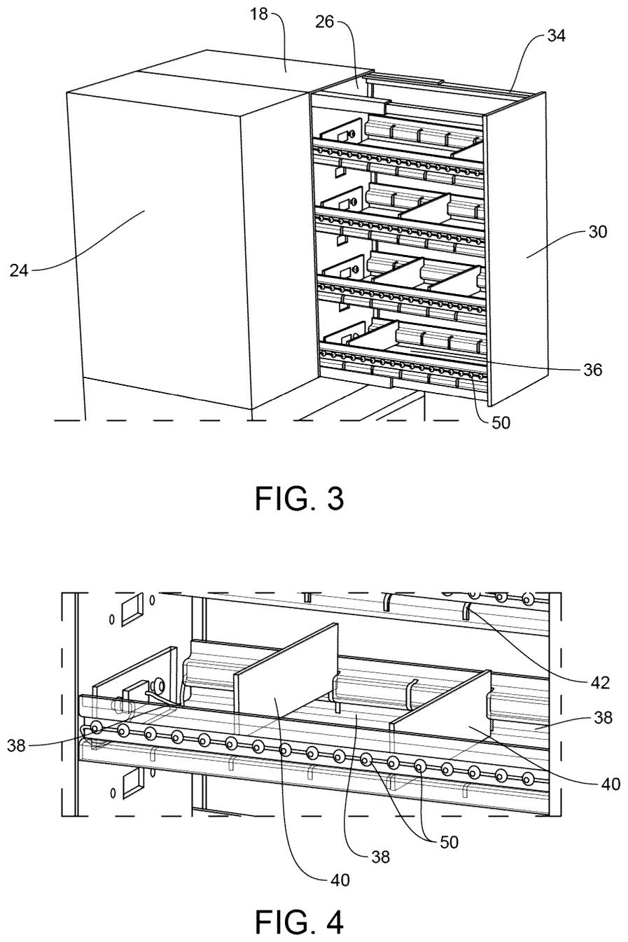

FIG. 3 provides an image of the upper storage unit 18 of a cabinet in an open condition. Accordingly, the unit 18 has an outer housing 24 defining a storage space 26 therein and having a front opening. A front panel 30 is interconnected to the outer housing 24 via a set of drawer sliders 34 that enable the front panel 30 to be moved in its entirety along a path of travel extending forward of the front opening from a closed position in which the front panel 30 closes the front opening (see FIG. 1) to an open position spaced forward of the front opening; (see FIG. 3).

A plurality of shelves 36 is integrated on a rear side of the front panel 30 at different heights such that, when the front panel 30 is in the closed position, the shelves 36 are housed entirely within the outer housing 26 and, when the front panel 30 is in the open position, the shelves 36 are located exterior of the outer housing 26. As best shown in FIG. 3, each of the shelves extends longitudinally relative to the path of travel of the front panel 30 and, when the front panel is in the open position, each of the plurality of shelves 36 are open and accessible along at least one or both sides of the path of travel. Each of the shelves 36 defines a plurality of compartments 38 in which medicine or medical supplies are stored.

Thus, each unit includes a single drawer having a plurality of shelves 36 that are located behind the front panel 30. The drawer comprises a linear drawer assembly that incorporates a plurality of shelves 36 each at a different height within the unit. Each shelf may contain an array of bins or compartments 38 for containing medications and/or other medical supplies. The compartments 38 are arranged one after the other behind the front panel 30.

The front panel 30 and integrated shelves 36 are supported on drawer sliders 34 located on all four corners of the front panel 30. Thus, the front panel 30 forms a front wall of the unit 18 and is not pivoted or supported by hinges. Raher, the front panel 30 moves straight outward such as a front panel of a drawer or filing cabinet, except that all the shelves 36 within the unit 18 are pulled outward with the front panel 30. In the illustrated embodiment, there are four shelves 36 per unit. Of course, the number and size of the shelves 36 can be altered as desired. The shelves 36 may be open on one or both sides of the shelves 36. If open on both sides, the compartments 38 may be accessed from either side of the shelves 36.

The front panel 30 and integrated shelves 36 as shown in FIG. 3 provide several advantages. A healthcare worker located in front of the cabinet 10 and shelves 36 has access to all shelves 36 within unit 18 with none being difficult to access. A part of the workspace or worksurface 22 or a bin 32 may be located adjacent the shelves 36 for easy access as discussed above. Thus, items can be readily removed from or replaced on the shelves 36.

As best illustrated in FIG. 4, the integrated shelves 36 may be configured in various configurations. The size of each compartment 38 on the shelf may be altered via use of dividers 40 supported in notches 42 or the like. The dividers 40 may be moved to a different set of notches 42 to make a compartment 38 larger or smaller as required. Thus, the end user is able to configure the various compartments 38 as desired. In addition, according to some embodiments, the multi-size compartments 38 may eliminate the requirement of having separate plastic bins for the items located and stored on the shelves 36. This can reduce cost and increase the flexibility of item placement.

Each of the shelves 36 as shown in FIGS. 3 and 4 include an array of lights 50, such as light emitting diodes (LEDs) or the like. The lights 50 are used to indicate the location of the selected medication or the like stored within the cabinet. For example, the medication inventory management software application program may control which of the lights 50 are illuminated which are not based upon the drug required for a particular patient. Thus, upon entering patient information or drug information for dispensing of a particular medication or the like, the inventory system will cause certain of the lights 50 to illuminate. Thus, the worker is readily informed of the particular shelf and the particular compartment on the shelf in which the required medication is located. Accordingly, the lights 50 are used to guide the worker to the correct compartment.

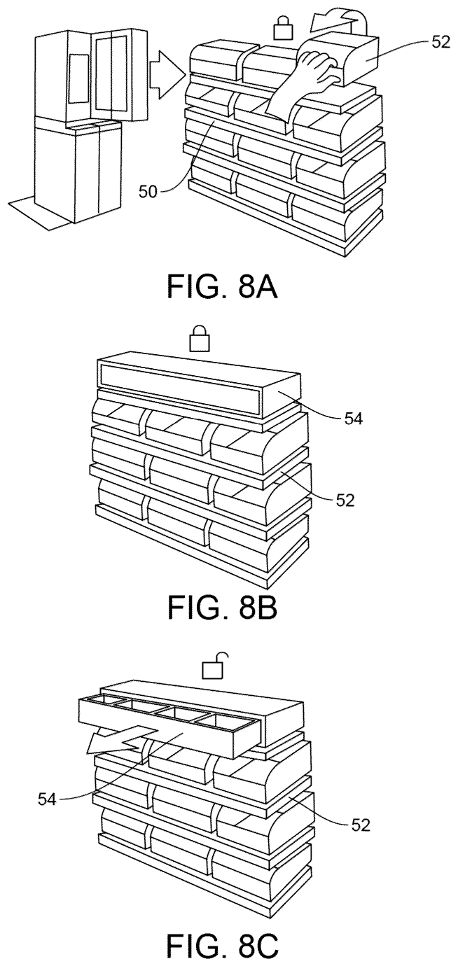

FIG. 8A discloses an alternate embodiment in which the compartments are provided on the shelves by a plurality of bins 52. The plurality of shelves of this embodiment also includes lights 50 that indicate the bin 52 containing the particular drug to be dispensed. In this embodiment there is one light per bin; however, fewer or more lights could be used. In addition, the light 50 may be lit in a steady state or blink to guide the worker to the correct bin 52. Also, instead of light, other forms of indication may be used, such as mechanically operated or other indicators.

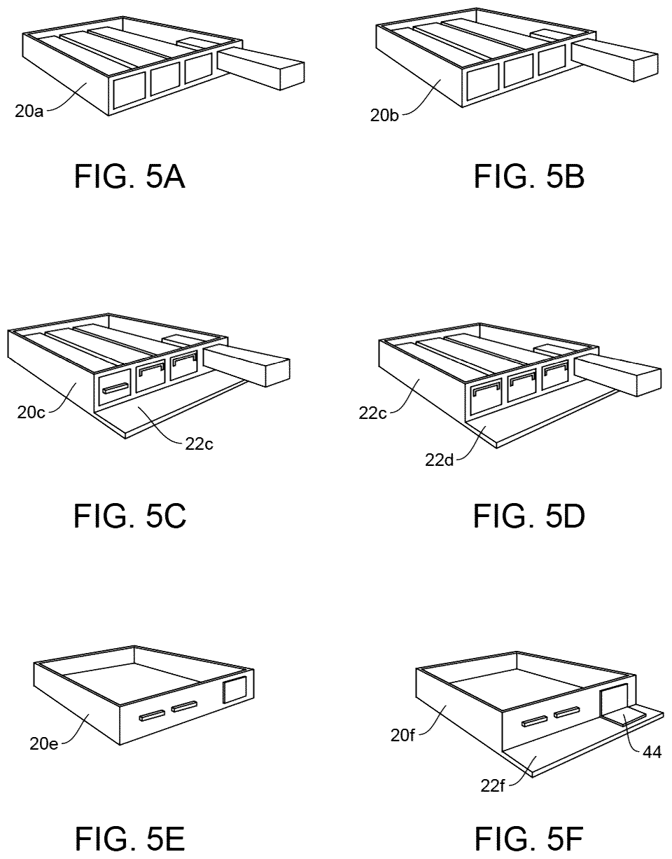

FIGS. 5A-5F disclose various embodiments of beltways or controller units for a cabinet. The various configurations enable the beltway of a cabinet to be configured as desired by the end user. The multiple configurations maximize flexibility. For instance, the beltway 20a of FIG. 5A includes four separate compartments and is without a front workspace or work surface. The compartments may include any arrangement of a kiosk style thermal printer or the like (i.e., for printing labels and/or receipts, a multi-read barcode scanner or the like, a limited access drawer (i.e., for narcotics or high value products), or the like. The beltway 20b of FIG. 5B may consist of limited access drawers only. The beltway 20c of FIG. 5C may be similar to beltway 20a, except beltway 20c may include an integral forwardly-extending work surface or workspace 22c. The beltway 20d of FIG. 20D may be similar to beltway 20b, except beltway 20d may include an integral forwardly-extending work surface or workspace 22d. The beltway 20e of FIG. 20E may include only a single relatively large compartment that is for various tech or electronic equipment only. The beltway 20f of FIG. 20F may be similar to beltway 20e, except beltway 20f may include an integral forwardly-extending work surface or workspace 22f having a built in scanner 44 (such as a barcode scanner or the like).

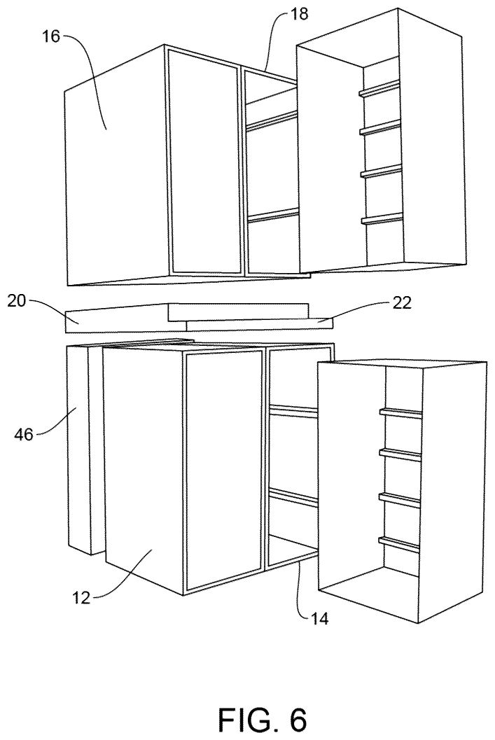

FIG. 6 shows the modular design of the cabinet according to embodiments which allow multiple configurations and sizes to be readily assembled. For instance, any number of lower or upper units can be combined together and a desired beltway may be incorporated therebetween. Each unit can be identical to all other units. In addition, a desired battery pack module 46 or the like can be configured behind the lower units.

The multiple configurations and sizes enable a desired cabinet to accommodate different types of practices based on inventory needs and/or space restrictions. Such cabinets are able to reduce the amount of unused storage space and reduce cost of equipment supplied to a customer. For instance, reversable handed drawer slides allows items to be access from either side of the shelves 36 as shown in FIG. 4 based upon room orientation and cabinet location.

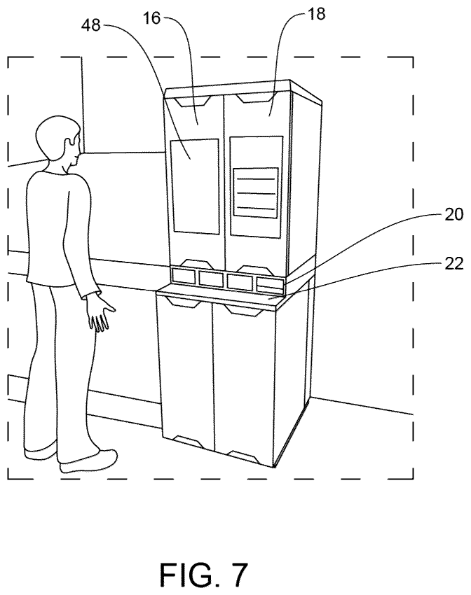

As shown in FIG. 7, the upper units and a beltway can be provided in a table-top version (i.e., without the use of lower units). Alternatively, the lower units can be replaced with a refrigerator unit or the like.

The embodiments of shelves in FIGS. 8B and 8C include bins 52 located on the shelves 36. In addition, one of the bins 54 may be lockable. For instance, bin 54 may be locking narcotic bin or the like. Thus, the bin 54 will remain locked as shown in FIG. 8B thereby preventing access to a worker have access to the cabinet. FIG. 8C shows the bin 54 in an unlocked condition permitting access to the contents of the bin 54. The inventory software can be programmed to permit this bin 54 to unlock based on pre-selected conditions.

The foregoing description and specific embodiments are merely illustrative of the principles thereof, and various modifications and additions may be made to the apparatus by those skilled in the art, without departing from the spirit and scope of this invention.

We claim:

1. A storage cabinet, comprising a plurality of storage units, each of said storage units including:
an outer housing defining a storage space therein and having a front opening;
a front panel interconnected to the outer housing via a set of drawer sliders that enable the front panel to be moved in its entirety along a path of travel extending forward of the front opening from a closed position in which the front panel closes the front opening to an open position spaced forward of the front opening; and
a plurality of shelves integrated with the front panel at different heights such that, when the front panel is in the closed position, the shelves are housed entirely within the outer housing and, when the front panel is in the open position, the shelves are located exterior of the outer housing;
each of the shelves extending longitudinally relative to the path of travel of the front panel and, when the front panel is in the open position, each of the plurality of shelves are open and accessible along at least one side of the path of travel; and
each of said shelves defining a plurality of compartments in which medicine or medical supplies are stored.

2. The storage cabinet according to claim 1, further comprising a controller unit located beneath or on top of said plurality of storage units, a height of said storage units being greater than a height of the controller unit.

3. The storage cabinet according to claim 2, wherein a pair of said storage units is located in proximity of said controller unit.

4. The storage cabinet according to claim 3, wherein a pair of said storage units is located beneath the controller unit.

5. The storage cabinet according to claim 2, wherein the controller unit includes an integral forwardly extending open work surface.

6. The storage cabinet according to claim 5, wherein the controller unit includes an electronic scanning device on or adjacent said work surface.

7. The storage cabinet according to claim 6, wherein the electronic scanning device is a barcode scanner.

8. The storage cabinet according to claim 2, wherein the controller unit includes at least one of a printer for printing labels or receipts, a barcode scanner, or a limited access drawer for narcotics or high value medical products.

9. The storage cabinet according to claim 1, wherein an array of lights is associated with each of said shelves such that illumination of one or more of the lights by an inventory management system indicates which one of the compartments contains a desired or needed medicine or medical supply.

10. The storage cabinet according to claim 1, wherein each of said front panels has an upper end with a recessed edge providing a hanging surface for a separate bin, and wherein each of said front panels includes said recessed edge on both said upper end and a lower end of said front panel.

11. The storage cabinet according to claim 1, wherein each of said shelves includes a plurality of dividers that define said compartments, wherein each of said dividers extends parallel to said front panel, and wherein a location of each of dividers is adjustable whereby a size of each of the compartments is adjustable.

12. The storage cabinet according to claim 11, wherein one or more lights is associated with each of said compartments such that illumination of one or more of the lights by an inventory management system indicates which one of the compartments contains a desired or needed medicine or medical supply.

13. The storage cabinet according to claim 1, wherein each of said shelves includes a plurality of bins that define said compartments, and wherein one or more lights is associated with each of said bins such that illumination of one or more of the lights by an inventory management system indicates which one of the bins contains a desired or needed medicine or medical supply.

14. The storage cabinet according to claim 1, wherein at least one said front panels includes a user interface of an inventory management system, and further comprising a battery unit positioned behind at least one of said storage units.

15. A medication storage cabinet for use with a computer controlled medication inventory management system for administering the inventory and distribution of medicines and medical supplies in a healthcare environment, comprising:
a plurality of storage units including a pair of upper storage units and a pair of lower storage units;
a controller unit supported on the pair of lower storage units and directly beneath the pair of upper storage units, said controller unit having a height less than a height of said storage units and including a forward extending open work surface; and
a battery unit located behind said pair of lower storage units such that said lower units extend forwardly of said upper storage units and underneath said work surface;
wherein at least one said front panels includes a user interface of the inventory management system.

16. The medication storage cabinet according to claim 15, wherein an array of lights is associated with each of said shelves such that illumination of one or more of the lights by the inventory management system indicates a location of a desired or needed medicine or medical supply.

17. The medical storage cabinet according to claim 15, wherein each of said plurality of identical storage units includes:
an outer housing defining a storage space therein and having a front opening;
a front panel interconnected to the outer housing via a set of drawer sliders that enable the front panel to be moved in its entirety along a path of travel extending forward of the front opening from a closed position in which the front panel closes the front opening to an open position spaced forward of the front opening; and
a plurality of shelves integrated with the front panel at different heights such that, when the front panel is in the closed position, the shelves are housed entirely within the outer housing and, when the front panel is in the open position, the shelves are located exterior of the outer housing;
each of the shelves extending longitudinally relative to the path of travel of the front panel; and
each of said shelves defining a plurality of compartments in which medicine or medical supplies are stored.

18. The storage cabinet according to claim 17, wherein an array of lights is associated with each of said shelves such that illumination of one or more of the lights by an inventory management system indicates which one of the compartments contains a desired or needed medicine or medical supply.

19. The medication storage cabinet according to claim 17, wherein the controller unit includes an electronic scanning device on or adjacent said work surface.

20. The medication storage cabinet according to claim 17, wherein the controller unit includes at least one of a printer for printing labels or receipts, a barcode scanner, or a limited access drawer for narcotics or high value medical products, and wherein each of said front panels of said lower storage units has an upper end with a recessed edge providing a hanging surface for a bin.

21. The medication storage cabinet according to claim 17, wherein each of said shelves includes a plurality of dividers that define said compartments, wherein each of said dividers extends parallel to said front panel, and wherein a location of each of dividers is adjustable whereby a size of each of the compartments is adjustable.

22. A medication storage cabinet for use with a computer controlled medication inventory management system for administering the inventory and distribution of medicines and medical supplies in a healthcare environment, comprising:
a plurality of storage units including a pair of upper storage units, at least one of said storage units including a user interface of the inventory management system;
a controller unit directly beneath the pair of upper storage units, said controller unit having a height less than a height of said storage units and including a forward extending open work surface and at least one of a printer for printing labels or receipts, a scanner, or a limited access drawer for narcotics or high value medical products; and
a plurality of lights within each storage such that illumination of one or more of the lights by the inventory management system indicates a location of a desired or needed medicine or medical supply within the storage unit.

23. A medication storage cabinet according to claim 22, wherein each of said plurality of identical storage units comprises:
an outer housing defining a storage space therein and having a front opening;
a front panel interconnected to the outer housing via a set of drawer sliders that enable the front panel to be moved in its entirety along a path of travel extending forward of the front opening from a closed position in which the front panel closes the front opening to an open position spaced forward of the front opening; and
a plurality of shelves integrated with front panel at different heights such that, when the front panel is in the closed position, the shelves are housed entirely within the outer housing and, when the front panel is in the open position, the shelves are located exterior of the outer housing;
each of the shelves extending longitudinally relative to the path of travel of the front panel; and
each of said shelves defining a plurality of compartments in which medicine or medical supplies are stored.
